SEQUENCE LISTING (20 sequences)

SEQ ID NO: 1 - DNA, 29 bp, Artificial Sequence
  Primer Sequences for Rat MOB-5 gene
atgcagacaagcttgagacaacagattct

SEQ ID NO: 2 - DNA, 27 bp, Artificial Sequence
  Primer Sequences for Rat MOB-5 gene
tcagagctggtagaaattctgcatcca

SEQ ID NO: 3 - DNA, 393 bp, Rattus
  prim_transcript (28)...(48): Gene Specific Primer Sequence designed from Rat MOB-5 gene
cttgattgccaatccggataacttcctcctttgttctccgtgccatttcaaggcattgttcatatccctgttgtcctcagggcacttcagacccttggccatggacccctgtcgttggctcaggcttttcttcagacctcactctttagtccaaacgacagccatggacagcacctttggatgctccgactgacccacaacgtggatttgcatatttattacagccctatttaactaatgtcactgtttcggtagaaaccggtatttatttgtgagactggacgttccatgaaagcatcatgccccgtgtttgcaccttacttcctgtgagctggctcaccatgggggcagtagatggttgctcagtaaatatttaaaatggaaaaaaaaaaa

SEQ ID NO: 4 - DNA, 21 bp, Artificial Sequence
  RT-PCR Primer region for rat MOB-5 gene
cctttgttctccgtgccattt

SEQ ID NO: 5 - DNA, 23 bp, Artificial Sequence
  RT-PCR Primer region for rat MOB-5 geneMOB-5 gene
ctttggatgctccgactgaccca

SEQ ID NO: 6 - protein, 183 aa, Rattus
  PEPTIDE (1)...(183): Amino Acid sequence corresponding to the Open Reading Frame region deduced from MOB-5 cDNAs Cloned from Rattus Dorsal Root Ganglia
MQTSLRQQILPGLSLILLVLSQVPELQGQEFRFGPCQVTGVVLPELWEAFWTVKNTVKTQDELTSVRLLKPQVLQNVSDAESCYLAHSLLKFYLNTVFKNYHSEIVKFKVLKSFSTLANNFLVIMSKLQPSKDNAMLPISDSARRRFLLFHRTFKQLDIEVALAKAFGEVDILLAWMQNFYQL

SEQ ID NO: 7 - DNA, 18 bp, Artificial Sequence
  Antisense Oligonucleotide
tcagcaggctgtgggcaa

SEQ ID NO: 8 - DNA, 18 bp, Artificial Sequence
  Antisense Oligonucleotide
tccgaaggcggtgtgcaa

SEQ ID NO: 9 - DNA, 18 bp, Artificial Sequence
  Antisense oligonucleotide
tcagcaggctgtgggcaa

SEQ ID NO: 10 - DNA, 18 bp, Artificial Sequence
  Antisense oligonucleotide
tccgaaggcggtgtgcaa

SEQ ID NO: 11 - DNA, 549 bp, Rattus
  gene (1)...(549): Rat MOB-5 cDNA
atgcagacaagcttgagacaacagattctccccggcctgagcctaatccttctcgttttgagccaagtaccagagcttcagggtcaagagttccgatttgggccttgccaagtgaccggggtggttctcccagaactgtgggaggccttctggactgtgaagaacactgtgaaaactcaggacgagctcacaagtgtccggctgttgaaaccacaggttctgcagaatgtctcggatgccgagagctgttaccttgcccacagcctgctgaagttctacttgaacactgttttcaaaaactatcacagcgaaatagtcaaattcaaggtcttgaagtcattctccactctggccaacaactttttagtcatcatgtccaaactgcagcctagtaaggacaatgccatgcttcccattagtgacagtgcacgccggcgttttttgctgttccacagaacattcaaacagttggacatagaagtggctttggcgaaagcctttggggaagtggacattctcctggcctggatgcagaatttctaccagctc

SEQ ID NO: 12 - DNA, 21 bp, Artificial Sequence
  Antisense Oligoribonucleotides sequence to rat MOB-5 gene
uucagcaggcugugggcaagg

SEQ ID NO: 13 - DNA, 21 bp, Artificial Sequence
  Sense Oligoribonucleotides sequence to rat MOB-5 gene
uugcccacagccugcugaatt

SEQ ID NO: 14 - DNA, 21 bp, Artificial Sequence
  Mismatch Antisense Oligoribonucleotides to rat MOB-5 gene
uuccgaaggcggugugcaagg

SEQ ID NO: 15 - DNA, 21 bp, Artificial Sequence
  Mismatch Sense oligoribonucleotides to rat MOB-5 gene
uugcacaccgccuucggaatt

SEQ ID NO: 16 - DNA, 18 bp, Artificial Sequence
  Antisense Oligoribonucleotides directed against rat MOB-5 gene
tcagcaggctgtgggcaa

SEQ ID NO: 17 - DNA, 18 bp, Artificial Sequence
  Antisense Oligoribonucleotides directed against rat MOB-5 gene
acagctctcggcatccga

SEQ ID NO: 18 - DNA, 18 bp, Artificial Sequence
  Antisense Oligoribonucletoides directed against rat MOB-5 gene
tcagcaggctgtgggcaa

SEQ ID NO: 19 - DNA, 18 bp, Artificial Sequence
  Antisense Oligoribonucleotides directed against rat MOB-5 gene
tccgaaggcggtgtgcaa

SEQ ID NO: 20 - DNA, 18 bp, Artificial Sequence
  Antisense Oligoribonucelotides directed against rat MOB-5 gene
ggccatccacagtcttct

(19) United States
(12) Patent Application Publication
(10) Pub. No.: US 2006/0128644 A1
(43) Pub. Date: Jun. 15, 2006

(54) Use of mob-5 in pain

(76) Inventors: Jane Barclay, London (GB); Francis Paul Buxton, Winchester, MA (US); Pamposh Ganju, London (GB); Francois Jean-Charles Natt, Aesch (CH); Chuanzheng Song, Southborough, MA (US); Jan Weiler, Lorrach-Haagen (DE); Willam Leonard Wishart, Allschwil (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/509,954

(22) PCT Filed: Apr. 2, 2004
(86) PCT No.: PCT/EP03/03469

§ 371 Date: Oct. 7, 2005

Related U.S. Application Data
(60) Provisional application No. 60/369,893, filed on Apr. 3, 2002.

(51) Int. Cl. A61K 48/00; A61K 39/395

(52) U.S. Cl. 514/44; 424/143.1

(57) ABSTRACT

The invention discloses Mob-5 as a suitable target for the development of new therapeutics to treat or ameliorate chronic pain. The invention relates to methods to treat and/or ameliorate chronic pain and pharmaceutical compositions therefor comprising modulators with inhibitory or stimulatory effect on Mob-5 activity and/or Mob-5 gene expression. The invention also relates to a method to identify compounds with therapeutic usefulness to treat chronic pain, comprising identifying compounds that can inhibit or stimulate Mob-5 activity and/or gene expression which can also reverse the pathological effects of chronic pain in vivo.

### BACKGROUND OF THE INVENTION

Pain is a term that encompasses a spectrum of clinical states. Under normal conditions acute pain is beneficial and serves as a physiological warning for a potentially tissue-damaging situation. More persistent pain, usually associated with inflammation, can also be regarded as a normal protective response to mild tissue injury and resolves when the injury has healed. However, chronic pain occurs when the stimulus and pain are unrelated and the pain is no longer a protective mechanism. These types of pain syndromes (e.g. rheumatoid arthritis, cancer pain, neuropathic pain) are notoriously difficult to treat. It is estimated that 10-20% of the adult population suffers from chronic pain. To date, the main analgesics employed are based on opiates and non-steroidal anti-inflammatory drugs (NSAIDS) such as aspirin. Both classes of drugs can produce severe side-effects; NSAIDS can cause gastric ulceration and renal damage while opiates can cause nausea, constipation, confusion and dependency problems. Despite these disadvantages, no new class of analgesics have been discovered or developed recently; there is clearly a need for additional therapies for chronic pain.

Chronic pain states are characterised by a number of clinical features. As well as spontaneous pain, patients may exhibit hyperalgesia (a greatly exaggerated response to a noxious mechanical, hot, or cold stimulus), and allodynia (previously non-noxious stimuli are now perceived as painful). All these features result from a complex series of events involving changes in the function of sensory nerves in the periphery and in the processing of sensory information in the spinal cord and brain. These changes occur in response to direct neuronal damage or in response to mediators released during tissue damage or inflammation.

Broadly speaking, chronic pain syndromes can be defined as inflammatory (also known as nociceptive) or neuropathic. Chronic inflammatory pain, as its name suggests, occurs during conditions in which there is underlying inflammation such as rheumatoid arthritis, burns, muscle damage or surgical wounds. Knowledge of the mechanisms underlying inflammatory pain has advanced considerably over recent years and it is known to involve a variety of mediators and their activation and sensitization of the peripheral terminals of sensory nerves and the consequent longer term changes in reactivity of spinal cord neurons.

Chronic neuropathic pain is caused where there is a primary lesion or dysfunction of the nervous system and occurs, for example, during conditions such as trigeminal neuralgia, diabetic neuropathy, post-herpetic neuralgia, amputation or physical nerve damage. Chronic neuropathic pain results from damage to nerves by trauma, by diseases such as diabetes, herpes zoster, or late-stage cancer (see below), or by chemical injury (e.g. some anti-HIV drugs). It may also develop after amputation (including mastectomy), and is involved in some low-back pain. The mechanisms of chronic neuropathic pain are poorly understood but are thought to involve spontaneous firing of sensory nerves due to the novel expression of certain classes of ion channel, sprouting of sensory fibres into different layers of the spinal cord, and changes in the expression of various neurotransmitters and receptors in the sensory nerves and spinal cord. Traditionally chronic neuropathic pain has proven to be intractable and is resistant to the standard non-steroidal and opiate analgesics. There is therefore clearly an unmet clinical need for new analgesics to treat this type of pain.

Cancer pain is the most common chronic pain syndrome (with probably inflammatory and neuropathic components). It is estimated that one third of patients with advanced cancer will develop skeletal metastases, particularly in breast, prostate and lung cancer. Metastatic bone disease commonly results in bone pain that is usually located to a discrete area and is described as a deep, boring sensation that aches and burns, accompanied by episodes of stabbing discomfort. The mechanisms responsible for bone cancer pain are unknown but it probably involves structural damage, periosteal irritation and nerve entrapment. There is evidence for the disruption of normal bone metabolism and the production of inflammatory prostaglandins and cytokines. Current treatment of bone cancer pain rests with opiates but the doses required results in unacceptable side-effects and at least 20% of patients still have uncontrolled pain. Novel, well tolerated and effective analgesics are desired to optimise the quality of life of these patients (Coleman RE (1997) Cancer80; 1588-1594).

Osteoarthritis pain is the most common form of chronic neuropathic pain (with probably inflammatory and neuropathic components) for which people visit general practitioners. Osteoarthritis is a chronic disease involving progressive structural changes in joint tissues, principally cartilage, synovium and subchondral bone. Typically, arthritic joints exhibit cartilage oedema and erosion, subchondral bone and synovial thickening, and formation of bony oesteophytes, all contributing to a deformation of the articular surface. The principal clinical symptom of osteoarthritis is pain, although the mechanisms underlying the chronic neuropathic pain in this condition are not understood.

Traditionally, attempts have been made to alleviate chronic neuropathic pain by directing therapeutic compounds to sensory fibers involved in pain signaling, e.g., the “C fiber”, (Woolf C. J. et al. (1995) J. Comp. Neurol. 360, 121-124.) or to the sensory fibers that transmit noxious information along the spinal cord (Dickenson A H. & Sullivan A. (1987) Neuropharmacol. 26; 1235-1238.). It has also been postulated that compounds may alleviate this pain by blocking mediator release (e.g. cytokines and bradykinins) from tissue during inflammation and/or blocking the receptors for these mediators (Dray A. & Urban L. (1996) Annu. Rev. Pharmacol. Toxicol. 36; 253-280.)

We have now discovered that mRNA for Mob-5, which encodes a secreted protein with homology to interleukins 10 and 20, is up regulated in animal models of chronic pain. Thus, Mob-5 can be used as novel drug target for chronic pain. It is also contemplated that the human ortholog of rat Mob-5, Interleukin 24 (IL24, NM 006850), which has 68% identical and 82% similar amino acids to rat Mob-5, is a similarly suitable target. The invention also provides a method for identifying modulators that inhibit or stimulate Mob-5 activity and/or inhibit or stimulate Mob-5 gene expression and the use of such modulators for the treatment of chronic pain in human and veterinary patients. The invention also provides pharmaceutical compositions comprising said modulators.

### SUMMARY OF THE INVENTION

The instant application relates to the discovery that Mob-5 is a suitable target for the development of new therapeutics to treat or ameliorate chronic pain, in particular chronic neuropathic pain. Thus, in one aspect the invention relates to a method to identify modulators useful to treat or ameliorate chronic pain, including chronic neuropathic pain, comprising: a) assaying for the ability of a candidate modulator to inhibit or stimulate the activity of Mob-5 and/or inhibit or stimulate Mob-5 gene expression in vitro or in vivo and which can further include b) assaying for the ability of an identified inhibitory modulator to reverse the pathological effects observed in animal models of chronic pain and/ or in clinical studies with subjects with chronic pain.

In another aspect, the invention relates to a method to treat or ameliorate chronic pain, including chronic neuropathic pain, comprising administering to a subject in need thereof an effective amount of a Mob-5 modulator, wherein said modulator, e.g., inhibits or stimulates the activity of Mob-5 or its receptor, and/or inhibits or stimulates Mob-5 or its receptor gene expression in said subject. In one embodiment the modulator comprises any one or more substances selected from the group consisting of antisense oligonucleotides, triple helix DNA, ribozymes, RNA aptamers, small-inhibitory RNA (siRNA) and double stranded RNA wherein said substances are designed to inhibit Mob-5 or receptor gene expression. In one embodiment, said Mob-5 modulator comprises one or more agonists to the Mob-5 receptor. In yet a further embodiment, said Mob-5 modulator comprises one or more antagonists to Mob-5 which may act directly on Mob-5 or may act at the receptor level. In one embodiment, said modulator comprises one or more antibodies to Mob-5, or fragments thereof, or to the Mob-5 receptor wherein said antibodies or fragments thereof can, e.g., inhibit Mob-5 activity.

In another aspect, the invention relates to a method to treat or ameliorate chronic pain, including chronic neuropathic pain, comprising administering to a subject in need thereof a pharmaceutical composition comprising an effective amount of a Mob-5 modulator. In various embodiments, said pharmaceutical composition comprises any of the Mob-5 modulators discussed above.

In another aspect, the invention relates to a pharmaceutical composition comprising a Mob-5 modulator in an amount effective to treat or ameliorate chronic pain, including chronic neuropathic pain, in a subject in need thereof wherein said modulator, e.g., can inhibit or stimulate the activity of Mob-5 or its receptor and/or inhibit or stimulate Mob-5 or receptor gene expression. In one embodiment, said pharmaceutical composition comprises any one or more substances selected from the group consisting of antisense oligonucleotides, triple helix DNA, ribozymes, RNA aptamers, siRNA or double stranded RNA directed to a nucleic acid sequence of Mob-5 wherein said substances are designed to inhibit Mob-5 or Mob-5 receptor gene expression. In yet a further embodiment, said pharmaceutical composition comprises one or more antagonists to Mob-5 which may act directly on Mob-5 or may act at the receptor level. In one embodiment, said pharmaceutical composition comprises one or more antibodies to Mob-5, or to the Mob-5 receptor or fragments thereof, wherein said antibodies or fragments thereof can, e.g., inhibit Mob-5 activity. In a further embodiment, said pharmaceutical composition comprises one or more agonists to the Mob-5 receptor.

In another aspect, the invention relates to a method to diagnose subjects suffering from chronic pain who may be suitable candidates for treatment with Mob-5 modulators comprising detecting levels of this protein and/or the Mob-5 receptor in a biological sample from said subject wherein subjects with increased levels compared to controls would be suitable candidates for Mob-5 modulator treatment.

In yet another aspect, the invention relates to a method to diagnose subjects suffering from chronic pain who may be suitable candidates for treatment with Mob-5 modulators comprising assaying mRNA levels of this protein and/or the Mob-5 receptor in a biological sample from said subject wherein subjects with increased levels compared to controls would be suitable candidates for Mob-5 modulator treatment.

In yet another aspect, there is provided a method to treat or ameliorate chronic pain, including chronic neuropathic pain, comprising: (a) assaying for Mob-5 and/or Mob-5 receptor mRNA and/or protein levels in a subject; and (b) administering to a subject with increased levels of Mob-5 and/or Mob-5 receptor mRNA and/or protein levels compared to controls a Mob-5 modulator in an amount sufficient to treat or ameliorate the pathological effects of chronic pain.

In yet another aspect of the present invention there are provided assay methods and kits comprising the components necessary to detect expression of polynucleotides encoding Mob-5, Mob-5 receptors, related regulatory polypeptides, or levels of Mob-5, receptors or related regulatory polypeptides, or fragments thereof, in body tissue samples derived from a patient, such kits comprising, e.g., antibodies that bind to said polypeptides, or to fragments thereof, or oligonucleotide probes that hybridize with said polynucleotides. In a preferred embodiment, such kits also comprise instructions detailing the procedures by which the kit components are to be used.

The present invention also pertains to the use of a Mob-5 modulator in the manufacture of a medicament for the treatment or amelioration of chronic pain, including chronic neuropathic pain. In one embodiment, said Mob-5 modulator comprises any one or more substances selected from the group consisting of antisense oligonucleotides, triple helix DNA, ribozymes, RNA aptamer, siRNA and double stranded RNA wherein said substances are designed to inhibit Mob-5 or receptor gene expression. In yet a further embodiment, said Mob-5 modulator comprises an antagonist to Mob-5 which may act directly on Mob-5 or may act at the receptor level. In one embodiment, said modulator comprises one or more antibodies to Mob-5, or to the Mob-5 receptor or fragments thereof, wherein said antibodies or fragments thereof can, e.g., inhibit Mob-5 activity. In another embodiment, said Mob-5 modulator comprises an agonist to the Mob-5 receptor.

The invention also pertains to a Mob-5 modulator for use as a pharmaceutical. In one embodiment, said Mob-5 modulator comprises any one or more substances selected from the group consisting of antisense oligonucleotides, triple helix DNA, ribozymes, RNA aptamer and double stranded RNA wherein said substances are designed to inhibit Mob-5 or receptor gene expression. In yet a further embodiment, said Mob-5 modulator comprises an antagonist to Mob-5 which may act directly on Mob-5 or may act at the receptor level. In one embodiment, said modulator comprises one or more antibodies to Mob-5, or to the Mob-5 receptor or fragments thereof, wherein said antibodies or fragments thereof can, e.g., inhibit Mob-5 activity. In another embodiment, said Mob-5 modulator comprises an agonist to the Mob-5 receptor.

### DETAILED DESCRIPTION OF THE INVENTION

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology are used. These techniques are well known and are explained in, for example, Harlow, E. and Lane, eds., 1988, “Antibodies: A Laboratory Manual”, Cold Spring Harbor Press, Cold Spring Harbor, Current Protocols in Molecular Biology, Volumes I, II, and IlIl, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and 11, 1985 (D. N. Glover ed.); Oligonucleotide Synthesis, 1984 (M. L. Gait ed.); Nucleic Acid Hybridization, 1985, (Hames and Higgins); Transcription and Translation, 1984 (Hames and Higgins eds.); Animal Cell Culture, 1986 (R. I. Freshney ed.); Immobilized Cells and Enzymes, 1986 (IRL Press); Perbal, 1984, A Practical Guide to Molecular Cloning; the series, Methods in Enzymology (Academic Press, Inc.); Gene Transfer Vectors for Mammalian Cells, 1987 (J. H. Miller and M. P. Calos eds., Cold Spring Harbor Laboratory); and Methods in Enzymology Vol. 154 and Vol. 155 (Wu and Grossman, and Wu, eds., respectively).

As used herein and in the appended claims, the singular forms “a”, “an”, and “the” include plural reference unless the context clearly dictates otherwise. Thus, for example, reference to the “antibody” is a reference to one or more antibodies and equivalents thereof known to those skilled in the art, and so forth.

The term “Mob-5” as used herein refers to Mob-5, Genbank # AAF75553 as well as the human ortholog of this protein, Interleukin 24 (Genbank # AAA91780). The human ortholog of rat Mob-5 is also known as hMDA-7 as well as “suppression of tumorigenicity 16 (Jiang, H et al., Oncogene 11, 2477-2486,1995) Included in the definition are any and all forms of these polypeptides including, but not limited to, variants, partial forms, isoforms, precursor forms, full length polypeptides, fusion proteins or fragments of any of the above, from human or any other species. Variants of Mob-5 include c49a Genbank, Accession Number NM; AAB69171. Homologs of Mob-5, which would be apparent to one of skill in the art, are also meant to be included in this definition. It is also contemplated that the term refers to Mob-5 isolated from naturally occurring sources of any species such as genomic DNA libraries as well as genetically engineered host cells comprising expression systems, or produced by chemical synthesis using, for instance, automated peptide synthesizers or a combination of such methods. Means for isolating and preparing such polypeptides are well understood in the art.

It has recently been discovered that the human counterpart of Mob 5, hMDA-7, binds to two types of heterodimeric IL-20R complexes: type I IL20R, composed of cytokine receptor IL-20Ralpha (GenBank Accession # NM_{—}014432) and IL-20Rbeta (GenBank Accession # AAZ20504), and type 11 IL20R, composed of IL-22Ralpha(GenBank Accession # AF286095) and IL20Rbeta. Data indicate that hMDA-7 signals through both complexes and binding of the ligand results in STAT3 phosphorylation and activation of a minimal promoter including STAT-binding sites. (Dumoutier, L. et al., J. Immunology, 2001, 167:3545-3549; Wang, M., Tan, Z., Zhang, R., Kotenko, S. V. and Liang, P. J. Biol. Chem. 2002, 277:7341-7347). Thus, as used herein, the term “Mob-5 receptor” includes both the human and rat forms of both the type I IL-20R and the type 11 IL-20R complexes.

“Pathological effects of chronic pain” include, but are not limited to, hyperalgesia and allodynia.

The ability of a substance to “modulate” Mob-5 (e.g., a Mob-5 modulator) includes, but is not limited to, the ability of a substance to inhibit or stimulate the activity of Mob-5, for example, by acting directly on Mob-5 or on the Mob-5 receptor , and/or by inhibiting or stimulating Mob-5 or Mob-5 receptor gene expression. Modulation could also involve effecting the ability of other proteins to interact directly (physical interaction) or indirectly (in the same signal transduction pathway) with Mob-5 or the Mob-5 receptor, for example related regulatory proteins or other proteins that are modified by/or modify Mob-5 or the Mob-5 receptor. Modulators include, but are not limited to, antagonists or inverse agonists of Mob-5 or the Mob-5 receptor, as well as antisense oligonucleotides, triple helix DNA, RNA aptamers, siRNA, ribozymes and double stranded RNA directed to a nucleic acid sequence of Mob-5 or to the Mob-5 receptor. It is also contemplated that a Mob-5 modulator may be a Mob-5 agonist.

The term “antagonist of Mob-5” as it is used herein refers to a molecule which decreases the amount or duration of the effect of the biological activity of Mob-5. Antagonists can include, but are not limited to, peptides, proteins, nucleic acids, carbohydrates, antibodies or any molecules which decrease the effect of Mob-5.

An “agonist” of Mob-5 refers to a substance that has affinity for and stimulates physiologic activity at the Mob-5 receptor and can thus trigger a biochemical response.

An “inverse agonist” is a substance that has affinity for the inactive form of a receptor and pushes the equilibrium to the active form but has the opposite effect of an agonist.

Based on recent findings that Mob-5 (IL24) signals through IL22Ralpha/IL20Rbeta and IL20Ralpha/IL20Rbeta complexes leading to the activation of STATs (Dumoutier, L. et al., J. Immunology, 2001, 167:3545-3549; Wang, M., Tan, Z., Zhang, R., Kotenko, S. V. and Liang, P. J. Biol. Chem. 2002, ,277:7341-7347), it is contemplated herein that the above mentioned antagonists or agonists could exert their effect, for example, by binding to these or similar receptor complex(es) or down stream components of the STAT signal transduction pathway.

“Nucleic acid sequence”, as used herein, refers to an oligonucleotide, nucleotide or polynucleotide, and fragments or portions thereof, and to DNA or RNA of genomic or synthetic origin that may be single or double stranded, and represent the sense or antisense strand.

The term “antisense” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines natural sequences produced by the cell to form duplexes. These duplexes then block either the further transcription or translation. The designation “negative ” is sometimes used in reference to the antisense strand, and “positive” is sometimes used in reference to the sense strand.

As contemplated herein, antisense oligonucleotides, triple helix DNA, RNA aptamers, siRNA, ribozymes and double stranded RNA are directed to a nucleic acid sequence of Mob-5 or the Mob-5 receptor such that the chosen nucleotide sequence of Mob-5 or receptor will produce gene-specific inhibition of Mob-5 or receptor gene expression. For example, knowledge of the Mob-5 nucleotide sequence may be used to design an antisense molecule which gives strong inhibition to the mRNA. Similarly, ribozymes can be synthesized to recognize specific nucleotide sequences of Mob-5 and cleave it (Cech. J. Amer. Med Assn. 260:3030 (1988). Techniques for the design of such molecules for use in targeted inhibition of gene expression is well known to one of skill in the art.

The term “sample” as used herein, is used in its broadest sense. A biological sample from a subject may comprise blood, urine or other biological material with which Mob-5 activity or gene expression may be assayed. A biological sample may include dorsal root ganglia from which total RNA may be purified for gene expression profiling using conventional glass chip microarray technologies such as Affymetrix chips, RT-PCR or other conventional methods.

As used herein, the term “antibody” refers to intact molecules as well as fragments thereof, such as Fa, F(ab′)₂, and Fv, which are capable of binding the epitopic determinant. Antibodies that bind Mob-5 polypeptides can be prepared using intact polypeptides or fragments containing small peptides of interest as the immunizing antigen. The polypeptides or peptides used to immunize an animal can be derived from the translation of RNA or synthesized chemically, and can be conjugated to a carrier protein, if desired. Commonly used carriers that are chemically coupled to peptides include bovine serum albumin and thyroglobulin. The coupled peptide is then used to immunize an animal (e.g., a mouse, a rat or a rabbit).

The term “humanized antibody” as used herein, refers to antibody molecules in which amino acids have been replaced in the non-antigen binding regions in order to more closely resemble a human antibody, while still retaining the original binding ability.

A “therapeutically effective amount” is the amount of drug sufficient to treat and/or ameliorate the pathological effects of chronic pain, including but not limited to, hyperalgesia.

“Related regulatory proteins” and “related regulatory polypeptides” as used herein refer to polypeptides involved in the regulation of Mob-5 or the Mob-5 receptor which may be identified by one of skill in the art using conventional methods such as described herein.

Pain as defined herein includes chronic pain. “Chronic pain” includes inflammatory (nociceptive) and neuropathic pain as described above.

“Subject” refers to any human or nonhuman organism.

The invention is based on the surprising discovery that Mob-5 messenger RNA is up regulated in rat models of chronic neuropathic pain. Thus, Mob-5 is a useful drug target for the development of therapeutics for the treatment of chronic pain, a disease state not previously known to involve Mob-5.

Thus, in one aspect the invention relates to a method to identify modulators useful to treat or ameliorate chronic pain, including chronic neuropathic pain, comprising: a) assaying for the ability of a candidate modulator to inhibit or stimulate the activity of Mob-5 and/or inhibit or stimulate Mob-5 gene expression in vitro or in vivo and which can further include b) assaying for the ability of an identified inhibitory/stimulatory modulator to reverse the pathological effects observed in animal models of chronic pain and/ or in clinical studies with subjects with chronic pain.

In addition, conventional techniques may be used to detect Mob-5 and Mob-5 receptor gene expression (e.g. mRNA levels). For example, conventional Northern analysis or commercially available microarrays may be used to quantitate mRNA levels. Additionally, the effect of test compounds on Mob-5, receptor and/or related regulatory protein levels can be detected with an ELISA antibody- based assay or fluorescent labelling reaction assay. These techniques are readily available for high throughput screening and are familiar to one skilled in the art.

Data gathered from these studies would be used to identify those modulators with therapeutic usefulness for the treatment of chronic pain as inhibitory/stimulatory substances could then be further assayed in conventional live animal models of chronic pain as described herein and/or in clinical trials with humans with chronic pain according to conventional methods to assess the ability of said compounds to ameliorate the pathological effects of chronic pain in vivo.

Candidate modulators for analysis according to the methods disclosed herein include chemical compounds known to possess Mob-5 modulatory activity as well as compounds whose effects on these proteins at any level have yet to be characterized. Compounds known to possess modulatory activity could be directly assayed in the animal pain models described herein or in clinical trials.

In another aspect, the invention relates to a method to treat or ameliorate chronic pain comprising administering to a subject in need thereof a pharmaceutical composition comprising an effective amount of a Mob-5 modulator. Such modulators include agonists, antagonists or antibodies directed to the Mob-5 polypeptide, Mob-5 receptor(s), or fragments thereof. In certain particularly preferred embodiments, the pharmaceutical composition comprises antibodies that are highly selective for human Mob-5 polypeptides/receptors or portions of human Mob-5 polypeptides/receptors. Antibodies to Mob-5 and/or to the Mob-5 receptor may cause the aggregation of these proteins in a subject and thus inhibit or reduce the activity of these proteins. Such antibodies may also inhibit or decrease Mob-5 activity, for example, by interacting directly with active sites or by blocking access of substrates to active sites.- Mob-5 and/or Mob-5 receptor antibodies may also be used to inhibit Mob-5 activity by preventing protein-protein interactions that may be involved in the regulation of Mob-5 and necessary for activity. Antibodies with inhibitory activity such as described herein can be produced and identified according to standard assays familiar to one of skill in the art.

Mob-5 antibodies may also be used diagnostically. For example, one could use these antibodies according to conventional methods to quantitate levels of Mob-5 in a subject; e.g. increased levels would indicate chronic pain and the degree of severity of this condition. Thus, different Mob-5 levels would be indicative of various clinical forms or severity of chronic pain. Such information would also be useful to identify subsets of patients experiencing pain that may or may not respond to treatment with Mob-5 modulators. Similarly, it is contemplated herein that quantitating the message level of Mob-5 in a subject would be useful for diagnosis and determining appropriate pain therapy; subjects with increased mRNA levels compared to appropriate control individuals would be considered suitable candidates for treatment with Mob-5 modulators.

Thus in another aspect, the present invention relates to a diagnostic kit which comprises:
(a) a polynucleotide of Mob-5 or a fragment thereof;
(b) a nucleotide sequence complementary to that of (a);
(c) a Mob-5 polypeptide, or a fragment thereof; or
(d) an antibody to a Mob-5 polypeptide.
   It will be appreciated that in any such kit, (a), (b), (c) or (d) may comprise a substantial component. It is also contemplated that said kit could comprise components (a)-(d) designed to detect levels of Mob-5 receptors, related regulatory proteins or proteins modified by Mob-5 as discussed herein.

Similarly, it is contemplated herein that monitoring Mob-5 protein levels or activity and/ or detecting Mob-5 gene expression (mRNA levels) may be used as part of a clinical testing procedure, for example, to determine the efficacy of a given pain treatment regimen. For example, patients to whom pain medicine has been administered would be evaluated and the clinician would be able to identify those patients in whom Mob-5 levels, activity and/or gene expression levels are higher than desired (i.e. levels greater than levels in control patients not experiencing pain or in patients in whom pain has been sufficiently alleviated by clinical intervention). Based on these data, the clinician could then adjust the dosage, administration regimen or type of pain medicine prescribed. While the clinician can get an idea of the effectiveness of a particular pain medication by asking the patient how much pain he or she is experiencing, it is contemplated herein that monitoring patient levels of Mob-5 as described above would provide a quantitative assessment of a patient's pain level. In addition, monitoring the level of Mob-5 in a subject in such a way could be used to assess the level of pain experienced by nonresponsive patients (e.g. infants, comatose, burn patients). Such data could then be used by the clinician for determining the appropriate dosage, administration regimen or type of pain medication for such patients.

Factors for consideration for optimizing a therapy for a patient include the particular condition being treated, the particular mammal being treated, the clinical condition of the individual patient, the site of delivery of the active compound, the particular type of the active compound, the method of administration, the scheduling of administration, and other factors known to medical practitioners. The therapeutically effective amount of an active compound to be administered will be governed by such considerations, and is the minimum amount necessary for the treatment of chronic pain, preferably, chronic neuropathic pain.

Suitable antibodies to Mob-5, the Mob-5 receptor, or related regulatory proteins may be obtained from a commercial source or produced according to conventional methods. For example, described herein are methods for the production of antibodies capable of specifically recognizing one or more differentially expressed gene epitopes. Such antibodies may include, but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-id) antibodies, and epitope-binding fragments of any of the above.

For the production of antibodies to Mob-5, the Mob-5 receptor or other polypeptides discussed herein, various host animals may be immunized by injection with the polypeptides, or a portion thereof. Such host animals may include, but are not limited to, rabbits, mice, and rats. Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as target gene product, or an antigenic functional derivative thereof. For the production of polyclonal antibodies, host animals such as those described above, may be immunized by injection with the polypeptides, or a portion thereof, supplemented with adjuvants as also described above.

Monoclonal antibodies, which are homogeneous populations of antibodies to a particular antigen, may be obtained by any technique which provides for the production of antibody molecules by continuous cell lines in culture. These include, but are not limited to the hybridoma technique of Kohler and Milstein, (1975, Nature 256:495-497; and U.S. Pat. No. 4,376,110), the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci., 81:6851-6855; Neuberger et al., 1984, Nature, 312:604-608; Takeda et al., 1985, Nature, 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable or hypervariable region derived from a murine mAb and a human immunoglobulin constant region.

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce differentially expressed gene-single chain antibodies. Single chain antibodies are formed by linking the heavy and light chain fragments of the Fv region via an amino acid bridge, resulting in a single chain polypeptide.

Most preferably, techniques useful for the production of “humanized antibodies” can be adapted to produce antibodies to the polypeptides, fragments, derivatives, and functional equivalents disclosed herein. Such techniques are disclosed in U.S. Pat. Nos. 5,932, 448; 5,693,762; 5,693,761; 5,585,089; 5,530,101; 5,910,771; 5,569,825; 5,625,126; 5,633,425; 5,789,650; 5,545,580; 5,661,016; and 5,770,429, the disclosures of all of which are incorporated by reference herein in their entirety.

Antibody fragments that recognize specific epitopes may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Detection of the antibodies described herein may be achieved using standard ELISA, FACS analysis, and standard imaging techniques used in vitro or in vivo. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, (3-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ³¹¹I ³⁵S or ³H.

Particularly preferred, for ease of detection, is the sandwich assay, of which a number of variations exist, all of which are intended to be encompassed by the present invention. For example, in a typical forward assay, unlabeled antibody is immobilized on a solid substrate and the sample to be tested brought into contact with the bound molecule. After a suitable period of incubation, for a period of time sufficient to allow formation of an antibody-antigen binary complex, a second antibody, labeled with a reporter molecule capable of inducing a detectable signal, is added and incubated, allowing time sufficient for the formation of a ternary complex of antibody-antigen-labeled antibody. Any unreacted material is then washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labeled antibody and sample to be tested are first combined, incubated and added to the unlabeled surface bound antibody. These techniques are well known to those skilled in the art, and the possibility of minor variations will be readily apparent. As used herein, “sandwich assay” is intended to encompass all variations on the basic two-site technique. For the immunoassays of the present invention, the only limiting factor is that the labeled antibody be an antibody which is specific for the Mob-5 polypeptide, receptor or related regulatory protein, or fragments thereof.

The most commonly used reporter molecules are either enzymes, fluorophore- or radionuclide-containing molecules. In the case of an enzyme immunoassay an enzyme is conjugated to the second antibody, usually by means of glutaraldehyde or periodate. As will be readily recognized, however, a wide variety of different ligation techniques exist, which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of polypeptide or polypeptide fragment of interest which is present in the serum sample.

Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed. It will be readily apparent to the skilled artisan how to vary the procedure to suit the required use.

The pharmaceutical compositions of the present invention may also comprise substances that inhibit the expression of Mob-5 or the Mob-5 receptor at the nucleic acid level. Such molecules include ribozymes, antisense oligonucleotides, triple helix DNA, RNA aptamers, siRNA and/or double stranded RNA directed to an appropriate nucleotide sequence of Mob-5 or receptor nucleic acid. These inhibitory molecules may be created using conventional techniques by one of skill in the art without undue burden or experimentation. For example, modifications (e.g. inhibition) of gene expression can be obtained by designing antisense molecules, DNA or RNA, to the important regions of the genes encoding the polypeptides discussed herein, For a mRNA of 3000 nucleotides, there are approximately 30000 different 10-20-mers possible. It is not possible to predict a priori how many and which of the 30000 possible 10-20-mer antisense oligonucleotides for a mRNA will have useful activity; experience shows that a small number will be active. The surest method to identify the best oligonucleotide would be to synthesize all 30000 sequences and test them individually. In practice this would be a huge effort. There are numerous reports in literature of a small selection of between 10-30 different antisense sequences providing suitable compounds. It would be helpful to be able to predict which regions of the target RNA are open to binding of oligonucleotides, however methods to determine whether a given region of a mRNA is accessible are limited. Computer programs to predict secondary interactions of RNA are available, but are relatively primitive and unable to give reliable predictions for long polynucleotides (e.g. greater than a few hundred nucleotides). The use of enzyme mapping experiments (Lima et al., Biochemistry 31: 12055-12061 (1992)) to reveal single and double-stranded regions of a RNA have been applied, but are tedious; multiple incubations with various enzymes and several polyacrylamide gels are required. Mapping of the RNA accessible regions with a combinatorial library of short DNA-oligonucleotides is a recently introduced technique (Lima et al., J. Biol. Chem. 272:626-638 (1997)). After hybridization of members of the library with the RNA, RNase H is introduced to give cleavage of the formed duplexes. These regions are subsequently identified on a polyacrylamide gel. This method is also tedious due to the limits of resolution of the gel, and use of the information is subject to the errors of supposing that single-stranded regions located by short oligonucleotides would also offer good binding sites to longer antisense sequences. Scanning array technology gives a direct readout of the binding capacity of a complete set of antisense oligonucleotides, and appears to be a big step forward in identifying accessible regions of a RNA target for high affinity duplex formation by antisense oligonucleotides (Southern et al., Nucleic Acids Res. 22: 1368-1373 (1994)). The method itself is quite straightforward and is described in detail in a number of peer-reviewed publications (see for example Southern et al., Nucleic Acids Res. 22: 1368-1373 (1994) and Nature Biotech. 15:537-541 (1997); Sohail et al., Molecular Cell Biology Research Communications 3: 67-72 (2000); WO 95/11748).

Once one or more target sites have been identified, oligonucleotides are chosen which are sufficiently complementary to the target, i.e., hybridize sufficiently well and with sufficient specificity, to give the desired effect, using, for example, any of the techniques mentioned above.

Similarly, inhibition of the expression of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

Ribozymes, enzymatic RNA molecules, may also be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences, for example, the gene for Mob-5.

Specific ribozyme cleavage sites within any potential RNA target are initially identified by scanning the target molecule for ribozyme cleavage sites which include the following sequences: GUA, GUU and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Ribozyme methods include exposing a cell to ribozymes or inducing expression in a cell of such small RNA ribozyme molecules (Grassi and Marini, 1996, Annals of Medicine 28: 499-510; Gibson, 1996, Cancer and Metastasis Reviews 15: 287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell (Cotten et al., 1989 EMBO J. 8:3861-3866). In particular, a ribozyme coding DNA sequence, designed according to conventional, well known rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. For saturating use, a highly and constituently active promoter can be used. tDNA genes (i.e., genes encoding tRNAs) are useful in this application because of their small size, high rate of transcription, and ubiquitous expression in different kinds of tissues.

Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be modified or perturbed.

Ribozyme sequences can be modified in essentially the same manner as described for antisense nucleotides, e.g., the ribozyme sequence can comprise a modified base moiety.

RNA aptamers can also be introduced into or expressed in a cell to modify RNA abundance or activity. RNA aptamers are specific RNA ligands for proteins, such as for Tat and Rev RNA (Good et al., 1997, Gene Therapy 4: 45-54) that can specifically inhibit their translation.

Double-stranded RNA (dsRNA) can also be used to inhibit gene expression by a mechanism generally known in the art as RNA interference (RNAi). RNAi is described for instance in U.S. Pat. No. 6,506,559 or in Patent Applications WO0244321 or WO0175164, the contents of which are herewith incorporated by reference. The length of the dsRNA is not crucial for RNAi according to the present invention, however, preferred dsRNAs are such which are generally known in the art as small-inhibitory RNAs (siRNAs). In a preferred embodiment, the siRNAs are short dsRNAs having a length of 19 to 25 nucleotides. Most preferred are dsRNAs having a length of 21 to 23 nucleotides. The dsRNAs may be blunt ended or ligated at or on at least one end with either loops composed of ribonucleotides or deoxyribonucleotides or a chemical synthetic linker (WO00/44895). In a preferred embodiment, the ribonucleic acid contains 3′-end nucleotide overhangs on the antisense strand and/or the sense strands of the dsRNA of at least one ribonucleotide or deoxyribonucleotide, or modified nucleotide. Preferred are overhangs with 1, 2, 3 or 4 nucleotides. The overhangs may contain both ribonucleotide(s) and deoxyribonucleotide(s) which in addition may contain modified sugar moieties. The overhang may be of any sequence, but in a preferred embodiment, the overhang is complementary to the target mRNA strand. In another preferred embodiment the overhang contains at least one UU group or dTdT group. In another preferred embodiment, the overhang on the antisense strand has the penultimate overhanging nucleotide complementary to the mRNA target strand. Preferably, such an overhang is a 2-nucleotides overhang. In a further preferred embodiment, the overhang is composed of 4 Us. In another preferred embodiment, the extreme 3′-position of the siRNA is a hydroxyl group. Additionally, the 5′-end may be a hydroxyl or phosphate group.

In related aspect, the present invention provides the use of on or more antisense or siRNA molecules that specifically inhibit the expression of Mob-5 genes for the manufacture of a medicament useful in the treatment of chronic pain.

Gene specific inhibition of gene expression may also be achieved using conventional double stranded RNA technologies. A description of such technology may be found in WO 99/32619 as well as Harborth J; et al., Journal of Cell Science (2001 December ), 114(Pt 24), 4557-65, and the entire contents of both references are hereby incorporated by reference.

Antisense molecules, triple helix DNA, RNA aptamers, ribozymes and double-stranded RNA of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

Vectors may be introduced into cells or tissues by many available means, and may be used in vivo, in vitro or ex vivo. For ex vivo therapy, vectors may be introduced into stem cells taken from the patient and clonally propagated for autologous transplant back into that same patient. Delivery by transfection and by liposome injections may be achieved using methods that are well known in the art.

In addition to the above described methods for inhibiting the gene expression of Mob-5 or the Mob-5 receptor, it is contemplated herein that one could identify and employ small molecules or other natural products to inhibit the transcription in vivo of the polypeptides discussed herein including, but not limited to, Mob-5. For example, one of skill in the art could establish an assay for Mob-5 that can be easily applied to samples from the culture media of a cell line using conventional methods. Using this assay, cell lines would be screened to find ones that express Mob-5. These cell lines would likely be of neuronal origin and would be cultured in, for example, 96 well plates. The closer the regulation of Mob-5 in the cell line to the expression in the dorsal root ganglia (DRG), the more likely it will be that small molecule modifiers of Mob-5 expression in the cell lines will also modify Mob-5 in DRG in vivo. A comparison of the effects of some known modifiers of gene expression e.g. dexamethasone, phorbol ester, heat shock on primary tissue DRG explants and the cell lines will allow the selection of the most appropriate cell line to use. The screen would then merely consist of culturing the cells for a set length of time with a different compound added to each well and then assaying for Mob-5 activity, protein levels, and/or mRNA level.

In order to facilitate the detection of Mob-5 in the assay described above, luciferase or other commercially available fluorescent protein could be genetically fused as an appropriate marker protein to the promoter of Mob-5. Sequences upstream of the ATG of Mob-5, i.e. the promoter of Mob-5, can be identified from genomic sequence data by using the sequence from GenBank accession number Mob-5 (Genbank # AAF75553) and c49a (Genbank # AAB69171), respectively hMDA-7 (Genbank # AAA91780) to BLAST against the NCBI genomic sequence. Two pairs of nested PCR primers to amplify a fragment of 2 kb or longer from human genomic DNA can be readily designed and tested. The promoter fragment can be readily inserted into any promoter-less reporter gene vector designed for expression in human cells (e.g. Clontech promoter-less enhanced fluorescent protein vector pECFP-1, pEGFP-1, or pEYFP). The screen would then consist of culturing the cells for an appropriate length of time with a different compound added to each well and then assaying for reporter gene activity. Promising compounds would then be assayed for effects on Mob-5 activity , protein levels, and/or mRNA levels in vivo using the in vivo models of chronic pain previously described. Additional method details such as appropriate culturing time, culture conditions, reporter assays and other methodologies that can be used to identify small molecules or other natural products useful to inhibit the transcription of Mob-5 in vivo would be familiar to one of skill in the art.

In addition, the cDNA and/or protein of Mob-5 can be used to identify other proteins, e.g. receptors, that are modified by Mob-5 in neurons from DRG or other tissues in the nervous system. Proteins thus identified can be used for drug screening to treat chronic pain. To identify these genes that are downstream of Mob-5, it is contemplated, for example, that one could use conventional methods to treat animals in chronic pain models with a specific Mob-5 inhibitor, sacrifice the animals, remove DRG and isolate total RNA from these cells and employ standard microarray assay technologies to identify message levels that are altered relative to a control animal (animal to whom no drug has been administered).

Based on the knowledge that Mob-5 is upregulated in chronic pain states, conventional in vitro or in vivo assays may be used to identify possible genes that lead to over expression of Mob-5. These related regulatory proteins encoded by genes thus identified can be used to screen drugs that might be potent therapeutics for the treatment of chronic pain. For example, a conventional reporter gene assay could be used in which the promoter region of Mob-5 is placed upstream of a reporter gene, the construct transfected into a suitable neuronal cell (for example, a neuroblastoma cell line) and using conventional techniques, the cells assayed for an upstream gene that causes activation of the Mob-5 promoter by detection of the expression of the reporter gene.

It is contemplated herein that one can inhibit the function and/or expression of a gene for a related regulatory protein or protein modified by Mob-5 as a way to treat chronic pain by designing, for example, antibodies to these proteins and/or designing inhibitory antisense oligonucleotides, triple helix DNA, ribozymes and RNA aptamers targeted to the genes for such proteins according to conventional methods. Pharmaceutical compositions comprising such inhibitory substances for the treatment of chronic pain are also contemplated.

The pharmaceutical compositions disclosed herein useful for treating and/or ameliorating chronic pain, including chronic neuropathic pain, are to be administered to a patient at therapeutically effective doses to treat or ameliorate symptoms of such disorders. A therapeutically effective dose refers to that amount of the compound sufficient to result in amelioration of pain symptoms of chronic pain based on, for example, use of the McGill pain score (Melzack, R. Pain (1975) September 1(3):277-299).

The inhibitory/stimulatory substances of the present invention can be administered as pharmaceutical compositions. Such pharmaceutical compositions for use in accordance with the present invention may be formulated in a conventional manner using one or more physiologically acceptable carriers or excipients.

Thus, the compounds and their physiologically acceptable salts and solvates may be formulated for administration by inhalation or insufflation (either through the mouth or the nose) or topical, oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Pharmaceutical compositions suitable for use in the invention include compositions wherein the active ingredients are contained in an effective amount to achieve the intended purpose. The determination of an effective dose is well within the capability of those skilled in the art.

For any compound, the therapeutically effective dose can be estimated initially either in cell culture assays, e.g., of neoplastic cells, or in animal models, usually mice, rabbits, dogs, or pigs. The animal model may also be used to determine the appropriate concentration range and route of administration. A dose may be formulated in animal models to achieve a circulating plasma concentration range that includes the IC₅₀ (i.e., the concentration of the test compound that achieves a half-maximal inhibition of symptoms). Such information can then be used to determine useful doses and routes for administration in humans.

A therapeutically effective dose refers to that amount of active ingredient, for example, antisense oligonucleotides, triple helix DNA, ribozymes, RNA aptamer and double stranded RNA designed to inhibit Mob-5 gene expression, antibodies to Mob-5 or related regulatory proteins or fragments thereof, useful to treat and/or ameliorate the pathological effects of chronic pain. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between toxic and therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc. Pharmaceutical formulations suitable for oral administration of proteins are described, e.g., in U.S. Pat. Nos. 5,008,114; 5,505,962; 5,641,515; 5,681,811; 5,700,486; 5,766,633; 5,792,451; 5,853,748; 5,972,387; 5,976,569; and 6,051,561.

Additionally, the present invention provides
(a) a package comprising a pharmaceutical composition described above and instructions for administering the pharmaceutical composition to treat or ameliorate chronic pain;
(b) a gene therapy vector comprising a nucleic acid molecule that encodes Mob-5 or a biologically active fragment thereof;
(c) a nucleic acid molecule that is complementary to a nucleic acid molecule that encodes Mob-5 or a fragment thereof;
(d) the use of Mob-5 or a biologically active fragment thereof in medicine;
(e) the use of a monoclonal antibody which specifically binds an epitope of Mob-5 or a biologically active fragment thereof in medicine; and
(f) the use of a compound that Mob-5 or has a stimulatory or inhibitory effect on the activity or the expression of Mob-5 for the manufacture of a medicament for the treatment of chronic pain.

The following examples further illustrate the present invention and are not intended to limit the invention.

### EXAMPLE 1

RNA Isolation and Expression Profiling of Mob-5 in Animal Models of Chronic Pain

In vivo animal models of chronic neuropathic pain include the following:

Seltzer Model.

In the Seltzer model (Seltzer et al. (1990) Pain 43: 205-218) rats are anaesthetised and a small incision made mid-way up one thigh (usually the left) to expose the sciatic nerve. The nerve is carefully cleared of surrounding connective tissues at a site near the trochanter just distal to the point at which the posterior biceps semitendinosus nerve branches off the common sciatic nerve. A 7-0 silk suture is inserted into the nerve with a ⅜ curved, reversed-cutting mini-needle, and tightly ligated so that the dorsal ⅓ to ½ of the nerve thickness is held within the ligature. The muscle and skin are closed with sutures and clips and the wound dusted with antibiotic powder. In sham animals the sciatic nerve is exposed but not ligated and the wound closed as in nonsham animals.

Chronic Constriction Injury (CCI) model.

In the CCI model ( Bennett, G. J. and Xie, Y. K. Pain (1988) 33: 87-107) rats are anaesthetised and a small incision is made mid-way up one thigh (usually the left) to expose the sciatic nerve. The nerve is cleared of surrounding connective tissue and four ligatures of 4/0 chromic gut are tied loosely around the nerve with approximately 1 mm between each, so that the ligatures just barely constrict the surface of the nerve. The wound is closed with sutures and clips as described above. In sham animals the sciatic nerve is exposed but not ligated and the wound closed as in nonsham animals.

Chung Model.

In contrast to the Seltzer and CCI models which involves damage to peripheral nerves, the Chung model involves ligation of the spinal nerve. (Kim, S. O. and Chung, J. M. Pain (1992): 50:355-363). In this model, rats are anesthetized and placed into a prone position and an incision is made to the left of the spine at the L4-S2 level. A deep dissection through the paraspinal muscles and separation of the muscles from the spinal processes at the L4-S2 level will reveal part of the sciatic nerve as it branches to form the L4, L5 and L6 spinal nerves. The L6 transverse process is carefully removed with a small rongeur enabling visualisation of these spinal nerves. The L5 spinal nerve is isolated and tightly ligated with 7-0 silk suture. The wound is closed with a single muscle suture (6-0 silk) and one or two skin closure clips and dusted with antibiotic powder. In sham animals the L5 nerve is exposed as before but not ligated and the wound closed as before.

Axotomy Model.

The Axotomy model involves complete cut and ligation of the sciatic nerve. The nerve endings form neuromas but there is no behavioral correlate in this model as the nerve is not allowed to regenerate, and the foot is permanently denervated. (Kingery and Vallin, Pain 38, 321-32,1989)

High Sciatic Lesion Model.

In this model, the sciatic nerve is punctured in the region of the iliac arch. Although there is no overt damage to the nerve, local swelling produces an increase in pressure on the nerve as it passes under the iliac arch. This model resembles conditions often seen in the clinic.

Chronic Inflammatory Pain Model:

The Complete Freund's Adjuvant -induced mechanical hyperalgesia may be used as a model of chronic inflammatory pain (Stein, C. et al. Pharmacol. Biochem. Behav. (1988) 31 :445-451). In this model, typically a male Sprague-Dawley or Wistar rat (200-250 9) receives an intraplantar injection of 25 μl complete Freund's adjuvant into one hind paw. A marked inflammation occurs in this hind paw. Drugs are generally administered for evaluation of efficacy, 24 hours after the inflammatory insult, when mechanical hyperalgesia is considered fully established.

Behavioral Index

In all chronic pain models (inflammatory and neuropathic) mechanical hyperalgesia is assessed by measuring paw withdrawal thresholds of both hindpaws to an increasing pressure stimulus using an Analgesymeter (Ugo-Basile, Milan). Mechanical allodynia is assessed by measuring withdrawal thresholds to non-noxious mechanical stimuli applied with von Frey hairs to the plantar surface of both hindpaws. Thermal hyperalgesia is assessed by measuring withdrawal latencies to a noxious thermal stimulus applied to the underside of each hindpaw. With all models, mechanical hyperalgesia and allodynia and thermal hyperalgesia develop within 1-3 days following surgery and persist for at least 50 days. For the assays described herein, drugs may be applied before and after surgery to assess their effect on the development of hyperalgesia, particularly approximately 14 days following surgery, to determine their ability to reverse established hyperalgesia.

The percentage reversal of hyperalgesia is calculated as follows:

$$\% reversal = \frac{postdose threshold - predose threshold}{naive threshold - predose threshold} \times 100$$

In the experiments disclosed herein, Wistar rats (male) are employed in pain models described above. Rats weigh approximately 120-140 grams at the time of surgery. All surgery is performed under enflurane/O₂ inhalation anaesthesia. In all cases the wound is closed after the procedure and the animal allowed to recover.

In all but the axotomy model, a marked mechanical and thermal hyperalgesia and allodynia develops in which there is a lowering of pain threshold and an enhanced reflex withdrawal response of the hind-paw to touch, pressure or thermal stimuli. After surgery the animals also exhibit characteristic changes to the affected paw. In the majority of animals the toes of the affected hind paw are held together and the foot turned slightly to one side; in some rats the toes are also curled under. The gait of the ligated rats varies, but limping is uncommon. Some rats are seen to raise the affected hind paw from the cage floor and to demonstrate an unusual rigid extension of the hind limb when held. The rats tend to be very sensitive to touch and may vocalise. Otherwise the general health and condition of the rats is good.

RNA extraction from DRG taken from rats subjected to chronic neuropathic pain models;

L4 and L5 DRG ipsilateral to the nerve injury are dissected from the rats at different times after surgery. Tissues are frozen in liquid nitrogen for subsequent total RNA preparation. Total RNA samples are prepared from the dissected DRG tissues according to the acid guanidinium thiocyanate-phenol-chloroform extraction method (Chomczynski and Sacchi Anal. Biochem., (1987) 162:156-159; Chomczynski, P., Biotechniques, 15: 532-537 (1993)). The yield is approximately 1 μg total RNA per DRG.

Sixteen RNA samples from rats subjected to the different chronic pain models were isolated at various time after surgery for Differential Display PCR (DD-PCR) analysis (Table 1).

TABLE 1
Total RNA samples for DD-PCR

| Samples # | Model | Days After Operation |
|---|---|---|
| 1 | Seltzer | 3 |
| 2 | Seltzer | 14 |
| 3 | Seltzer | 21 |
| 4 | Seltzer | 50 |
| 5 | Sham | 3 |
| 6 | Sham | 14 |
| 7 | Sham | 21 |
| 8 | Sham | 50 |
| 9 | Axotomy | 14 |
| 10 | Chung (L4) | 28 |
| 11 | Chung (L5) | 28 |
| 12 | Chung Sham | 28 |
| 13 | CCI | 14 |
| 14 | CCI | 21 |
| 15 | High Sciatic | 14 |
| 16 | High Sciatic | 21 |

DD-PCR, band isolation and subcloning:

The RNAimage™ mRNA Differential Display system from GenHunter Corp. (Nashville, Tenn.) is used for DD-PCR analysis of the 16 RNA samples. The reverse transcription reaction is performed according to the manufacturer's protocal except a mixture of equal parts MMLV reverse transcriptase (GenHunter, Nashville, Tenn.) and Superscript II reverse transcriptase (Invitrogen, Carlsbad, Calif.) is usded. PCR amplification of the cDNA population is performed according to the manufacturer's instructions, using each of the 80 arbitrary primers with each one-base anchored oligo-dT primer and the similarly primed cDNA template. The PCR products are labelled with ³³P-dATP and resolved on denaturing 6% urea-polyacrylamide gels with two sets of 16 PCR reactions loaded per gel. The combination of 80 rationally designed 1 3-mers and 3 one-base anchored oligo(dT)₁₁ allow 240 PCR reactions. Bands that are consistently up- or down-regulated in all the five models are isolated, re-amplified using the same primer pair used for DD-PCR and the PCR products subcloned into pCR4-TOPO vector through topoisomerase mediated TA cloning strategy (Invitrogen, Carlsbad, Calif.). Plasmid DNA is then prepared using QlAprep miniprep kit (QIAGEN, Valencia, Calif.) and sequenced using theT7 primer.

Confirmation of Regulated Genes by RT-PCR:

One ug of total RNA is treated with 0.1 unit of RNase-free DNase (Roche Mol.Biochem Indianapolis, Ind.,) at 37° C. for 5 minutes and used for RT-PCR with primers designed from the sequences of the cloned cDNA fragments, according to conventional methods.

PCR products are analyzed on a 4-20% TBE Criterion polyacrylamide gel (BioRad, Hercules, Calif.,), stained with SYBR Green I (Molecular Probes, Eugene, Oreg.) and visualized using a Phosphorimager (Molecular Dynamics, Sunnyvale, Calif.).

Molecular Cloning and Expression of Mob-5:

The following primers are used to amplify the coding sequence of rat Mob-5 by RT-PCR from total RNA samples isolated on Day 14 from DRG from rats subjected to the Seltzer model.

SEQ ID NO: 1

DD10Forward: ATG CAG ACA AGC TTG AGA CAA CAG ATT
CT

SEQ ID NO: 2

DD10Reverse.: TCA GAG CTG GTA GAA ATT CTG CAT CCA

PCR products were cloned into the pCR4-TOPO vector (Invitrogen, Carlsbad, Calif.) and sequenced according to conventional methods.

### EXAMPLE 2

Mob-5 mRNA is Upregulated in Rat DRG from Chronic Pain Models

Examination of 240 sets of autoradiograms generated by the labeled DD-PCR products resulted in a total of 40 bands that are upregulated in at least two DRG RNA samples from the animal models; no down-regulated products are identified (data not shown) Two bands, #10 and #41, are consistently upregulated in DRG samples from all five models (Table 2).

TABLE 2
Identification of a gene upregulated in all neuropathic pain models

| | | Identical | RT-PCR | |
|---|---|---|---|---|
| Band | Size (bp) | clones | confirmation | Gene |
| 10 | 393 | 9 | yes | Mob-5 |
| 41 | 243 | 5 | yes | Mob-5 |

Sequence analysis of the cloned PCR products indicate that bands 10 and 41 are from the same transcript, which is identical to rat gene Mob-5 (Zhang et al, J. Biol. Chem. 2000; 275: 24436-24443).

Gene-specific primers (Table 3) are designed from the Mob- 5 sequence and used for RT-PCR confirmation from selected DRG total RNA samples according to conventional methods.

PCR products are analyzed on 4-20% TBE polyacrylamide gels stained with SYBR green. Data indicate that the Mob-5 gene has about 2-5 fold more RNA accumulation in DRG samples from rats in Chung, CCI, Seltzer and axotomy models, compared to sham, in agreement with the banding pattern seen on the original differential display gels (data not shown).

TABLE 3

Mob-5 contig composed of 14 sequences from bands
10 and 41 that give rise to upregulated DD-PCR
products

CTTGATTGCC AATCCGGATA ACTTCCTCCT TTGTTCTCCG
TGCCATTTCA AGGCATTGTT CATATCCCTG TTGTCCTCAG
GGCACTTCAG ACCCTTGGGC ATGGACCCCT GTCGTTGGCT
CAGGCTTTTC TTCAGACCTC ACTCTTTAGT CCAAACGACA
GCCATGGACA GCACCTTTGG ATGCTCCGAC TGACCCACAA
CGTGGATTTG CATATTTATT ACAGCCCTAT TTAACTAATG
TCACTGTTTC GGTAGAAACC GGTATTTATT TGTGAGACTG
GACGTTCCAT GAAAGCATCA TGCCCCGTGT TTGCACCTTA
CTTCCTGTGA GCTGGCTCAC CATGGGGGCA GTAGATGGTT
GCTCAGTAAA TATTTAAAAT GGAAAAAAAA AAA SEQ ID NO: 3

The regions used for designing gene-specific primers for RT-PCR confirmation are in bold-face and underlined.

| | |
|---|---|
| CCT TTGTTCTCCG TGCCATTT | SEQ ID NO:4 |
| CTTTGG ATGCTCCGAC TGACCCA | SEQ ID NO:5 |

### EXAMPLE 3

Molecular Characterization of Mob-5 Transcripts From Rat DRG

Rat Mob-5 has been identified as a novel interleukin 10-like molecule induced during wound repair (Soo et al., J. Cell. Biochem. 1999; 74:1-10) and induced by ras oncogenes (Zhang et al. J. Biol. Chem. 2000; 275: 24436-24443) and its cDNA was cloned from rat embryo fibroblast and epithelial cells. Based on the published sequence (AAF75553) we cloned the ORF region (encoding a peptide of 183 amino acids) of Mob-5 cDNA by RT-PCR from rat total DRG RNAs from the Seltzer model at day 14 using conventional methods. The deduced amino acid sequence of Mob-5 cloned from rat DRG is shown in Table 4 and is slightly different from the published Mob-5 sequence (GenBank # MF75553) at positions 21 (N to S), 104 (K to E) and 150 (Y to F).

Conventional Northern blot analysis of mRNA samples from 12 normal adult rat tissues using radioactive probes synthesized from the cloned Mob-5 cDNA reveal a weak band around 1.2 kb in testis, spleen and thymus (data not shown).

TABLE 4
Amino Acid Sequences of Mob-5 Deduced from cDNAs Cloned From DRG

| | |
|---|---|
| MQTSLRQQIL PGLSLILLVL SQVPELQGQE FRFGPCQVTG VVLPELWEAF | 50 |
| WTVKNTVKTQ DELTSVRLLK PQVLQNVSDA ESCYLAHSLL KFYLNTVFKN | 100 |
| YHSEIVKFKV LKSFSTLANN FLVIMSKLQP SKDNAMLPIS DSARRRFLLF | 150 |
| HRTFKQLDIE VALAKAFGEV DILLAWMQNF YQL | 183 |
| SEQ ID NO: 6 | |

### EXAMPLE 4

Antisense Oligonucleotides to Mob-5

Synthesis of Antisense Oligosnucleotides:

Antisense oligonucleotides (ASOs) useful to inhibit gene expression, including the expression of Mob-5 or the Mob-5 receptor, may be made according to conventional methods. For example, ASOs against Mob-5 in vitro may be fully or partially phosphorothioated phosphodiesterl8-mers with nucleotides at both ends modified with MOE (methoxy ethoxy) groups. For example, ASOs against Mob-5 in vivo may be partially phosphorothioated or fully phosphodiester-18-mers with nucleotides at both ends modified with MOE (methoxy ethoxy) groups. These may be synthesized using phosphoramidite chemistry, HPLC-purified and characterized by electrospray mass spectrometry and capillary gel electrophoresis according to conventional methods. ASOs, each with a GC content between 38 and 72%, may be selected and synthesized complementary to parts of the coding region of, for example, rat Mob-5 or human IL24 (hMDA-7). For mismatch-containing control oligonucleotides, the approximate base composition of the match oligonucleotides may be maintained. Additionally, two control ASOs may be selected, e.g., one for rat GAPDH coding regions and a second random synthetic ASO. The format of the anti-rat-GAPDH oligonucleotide may be the same as for anti- Mob-5 oligonucleotides; the synthetic oligonucleotide may have its MOE ribonucleotide modifications at both ends of the sequence with phosphorothioate or phosphodiester DNA residues in the middle.

In vitro selection of Mob5 ASOs:

Using methods familiar to one of skill in the art, optimal ASOs can be selected from a collection of ASO candidates in vitro in order to identify the most active ASO for subsequent analyses (e.g. in vivo target validation). Such ASO candidates can be tested in comparison with mismatched ASOs (MSOs) (i.e. otherwise identical ASOs bearing conservative inactivating mutations), vehicle, and/or untreated controls. Once an optimal candidate ASO sequence has been identified as a target for antisense, chemical derivatives and formats, more suited for in vivo applications, and based on the identical optimal target sequence, can then be synthesized and subsequently administered in vivo.

Transfection Protocol:

Transfection of ASOs may be performed according to methods familiar to one of skill in the art. For example, twenty four hours before transfection, 2×10⁵ cells e.g., ATCC CRL-1747 (ATCC, Manassas, Va.) in a volume of 2 ml per well (F12 Nutrient mix (DMEM), 100 unit/millilitre Penicillin, 100 micrograms per millilitre streptomycin, 2 millimolar L-Glutamine, 10% fetal bovine serum (GIBCO-BRL, Rockville, Md.)) may be plated into 6-well plates and cultured in 5% CO₂ to yield 70-80% confluency. On the day of transfection, a 2 fold stock transfection solution is prepared by diluting Lipofectin™ into serum-free OptiMEM (GIBCO-BRL, Rockville, Md.) (3 microliters Lipofectin™ per 100 nM desired final oligonucleotide concentration into 1 ml OptiMEM) and incubating for 15 minutes at room temperature. This solution is then combined 1:1 with a 2 fold ASO-solution containing twice the desired final amount of ASO in OptiMEM. After incubating the transfection mixture for 15 minutes at room temperature to form the transfection complex, 2 ml is added to each of the previously aspirated well of cells. A Lipofectin™ reagent-only control and a normal cell control (untreated) may also be included. After incubation for 4 hours at 37° C., 500 microlitres of 50% FBS in MEM (Invitrogen, Carlsbad, Calif.) is then added to each well to obtain a final FBS concentration of 10%. The cultures are then incubated at 37° C. in a humidified incubator with 5% CO₂ for 24 hours for mRNA harvest or 48 hours for protein harvest and electrophysiology.

Real-Time Quantitative PCR mRNA Analysis:

Real-time quantitative PCR mRNA analysis may be performed according to methods standard in the art. For example, total RNA may be isolated with the RNeasy 96 Kit (Qiagen, GmBH, Germany) according to the manufacturer's protocol. The RNA samples are individually diluted to 1 ng/L. Five nanograms of RNA for each sample are then mixed with gene-specific detection primers (easily determined by one of skill in the art) and with the appropriate reagents from the real-time quantitative PCR reaction kit PLATINUM® Quantitative RT-PCR THERMOSCRIPT™ One-Step System (Gibco-BRL, Rockville, Md.) and run according to manufacturer's protocol. The rat Mob-5 primers with the appropriate sequences may be purchased from PE Applied Biosystems, (Foster City, Calif.). GAPDH may be chosen as a control gene for comparisons. The same RNA samples may be run with rat GAPDH primers from the TaqMan® Rodent GAPDH Control Reagents Kit (PE Biosystems). The sequence-specific fluorescent emission signal can be detected using the ABI PRISM™7700 Sequence Detector (PE Applied Biosystems, Foster City, Calif.). Along with the samples, a standard from dilutions of pure template mRNA is run to obtain absolute concentrations per inserted amount of total RNA.

MobS RNA analysis In vitro: Dose Response vs. Mismatch:

Fourteen Mob-5 specific ASOs are synthesized, along with mismatch controls each bearing 4 mutations compared with the original match ASOs. Briefly, phosphorothioated 18-mers with 5 nucleotides at the 3′ and 5′-ends modified with 2′-O-(2-methoxyethyl) (MOE) groups, are synthesized using phosphoramidite chemistry (Martin, P and Natt, F. EP 0 992 506 ), HPLC-purified, and characterised by electrospray mass spectrometry and capillary gel electrophoresis. In vitro selection is then performed as described above.

The ASO which inhibited Mob-5 mRNA levels the best, as determined by real time quantitative PCR in an in vitro assay performed on the cell line ATCC CRL-1 747 (which expresses relatively high levels of endogenous Mob-5 mRNA) was ASO 7429 (SEQ ID NO: 7 ) which was then tested against mismatch control MSO 7443 (SEQ ID NO: 8) in a dose response experiment again using CRL1747 cells. The IC50 for 7429 is approximately 100 nM.

The dose response against mismatch results are included in Table 5 below and indicate that ASO 7429 can inhibit Mob-5 RNA levels by >80% at concentrations of 200 nM and higher:

TABLE 5
(*p-values<001 against Lipofectin, Mismatch, and Untreated controls:
average values expressed in fg Mob5 mRNA/50 ng total cellular RNA)

| ASO Treatment Avg. Value | Std. Error |
|---|---|
| 7429 400 nM* | 173.33 ± 6.63 |
| 7429 200 nM* | 196.67 ± 71.39 |
| 7429 100 nM | 463.33 ± 73.89 |
| 7429 50 nM | 593.33 ± 87.78 |
| 7429 12.5 nM | 1090.00 ± 205.69 |
| 7443 400 nM | 990.00 ± 218.31 |
| 7443 200 nM | 1206.67 ± 202.48 |
| 7443 100 nM | 973.33 ± 311.90 |
| 744 350 nM | 676.67 ± 174.91 |
| 7443 12.5 nM | 1163.33 ± 262.13 |
| Lipofectin | 753.33 ± 124.90 |
| Untreated | 1003.33 ± 96.22 |

Based on the dose response data in vitro above, a nonphosphorothioated version of ASO 7429, referred to as ASO 8154 (SEQ ID NO: 9) and a nonphosphorothioated version of missense oligonucleotide MSO 7443, referred to as MSO 8155 (SEQ ID NO: 10), is used in vivo (see below).

SEQ ID NO: 7

ASO 7429 tcagcAsGsGsCsTsGsTsGsggcaa

SEQ ID NO: 8

MSO 7443 tccgaAsGsGsCsGsGsTsGstgcaa

SEQ ID NO: 9

ASO 8154 tcagcAGGCTGTGggcaa

SEQ ID NO: 10

MSO 8155 tccgaAGGCGGTGtgcaa

Lowercase (a, t, g) indicates nucleotides modified with 2′- MOE groups and c refers to 5-methyl-cytosine modified with 2′- MOE group, and “s” indicates a phosphorothioate group.
Effect of Mob 5 ASOs on Rats in the Seltzer Model of Chronic Pain:

In order to evaluate the effect of ASOs or MSOs in vivo, for example, rats (e.g. Wistar) may be intrathecally cannulated in the lumbar or thoracic region of the spinal cord with a catheter attached to a minipump delivery system according to conventional methods. Antisense, missense oligos or vehicle may then be delivered for up to 7 days at a desired concentration to allow cell bodies within the spinal cord and the dorsal root ganglia to take up the oligos or vehicle. Nerve injury may be performed either before or after cannulation according to the pain models described herein. Mechanical hyperalgesia, allodynia etc may be measured according to conventional methods to assess the effect of Mob-5 antisense oligonucleotides in reversal of hyperalgesia.

In this case, the partial sciatic ligation (Seltzer) model of neuropathic pain is used as previously described (Seltzer Z, Dubner R, Shir Y ,1990, Pain 43:205-218). Briefly, male Wistar rats (120-140 g) are anaesthetised, the left sciatic nerve exposed at mid-thigh level through a small incision and ⅓ to ½ of the nerve thickness tightly ligated with a 7.0 silk suture. The wound is closed with sutures and clips and the wound dusted with antibiotic powder. In sham animals the sciatic nerve is exposed but not ligated and the wound closed as before. Mechanical hyperalgesia is assessed by measuring paw withdrawal thresholds of both hindpaws to an increasing pressure stimulus using an Analgesymeter (Ugo-Basile, Milan). The cut-off is set at 250 g and the end-point taken as paw withdrawal, vocalisation or overt struggling. The statistical significance of mechanical hyperalgesia data obtained from the different experimental animal groups is analysed using ANOVA followed by Tukey's HSD test. All experiments are carried out according to Home Office (United Kingdom) guidelines and with approval of the local Novartis Animal Welfare and Ethics Committee.

Antisense oligonucleotide ASO-8154 and mismatch oligos MSO-8155 are administered to rats intrathecally (180 μg/ml) via an indwelling cannula, inserted 24 h prior to or 14 days following sciatic nerve ligation, (or 24 h prior to injection of 25 pl Complete Freund's Adjuvant into a hindpaw if one uses an inflammatory pain model). Rats are anaesthetised and an incision made in the dorsal skin just lateral to the midline and approximately 10 mm caudal to the ventral iliac spines. A sterile catheter (polyethylene PE10 tubing) is inserted via a guide cannula (20 gauge needle) and advanced 3 cm cranially in the intrathecal space approximately to the L1 level. The catheter is then connected to an Alzet mini osmotic-pump (Alza Corporation, Palo Alto, Calif.) delivering Mob-5 ASO, mismatch oligo or saline (1 μl /h, 7 days) which is inserted subcutaneously in the left or right flank. The incision is closed with wound clips and dusted with antibiotic powder. Preliminary experiments determined 180 μg/day as a maximal tolerated dose. Delivery of ASO to the DRG cell bodies is initially confirmed using a fluorescently-labelled ASO, an unlabelled version is used in all subsequent experiments.

Results indicate that the intrathecal administration of Mob-5 ASO over 7 days on established neuropathic mechanical hyperalgesia in rats resulted in a significant increase in withdrawal threshold in the paw pressure test, representative of decreased mechanical hyperalgesia (Table 6). This effect was significant for four days and was not seen following similar administration of MSO or saline (vehicle). These data confirm that modulators of Mob-5 or its receptor are useful in blocking chronic neuropathic pain.

TABLE 6
Mean withdrawal threshold (g) ± SEM, taken from left (ligated) paw

| | Time after Cannulation (days) | | | | | |
|---|---|---|---|---|---|---|
| Treatment | 0 | 1 | 2 | 3 | 4 | 5 |
| Vehicle | 63.3 ± 1.1 | 66.7 ± 1.1 | 63.3 ± 1.8 | 60.0 ± 2.0 | 66.7 ± 1.8 | 64.2 ± 1.7 |
| ASO | 64.2 ± 1.7 | 71.7 ± 3.4 | 82.5 ± 3.1* | 79.1 ± 2.6* | 82.5 ± 2.7* | 77.5 ± 4.6* |
| MSO | 64.2 ± 1.7 | 65.0 ± 1.7 | 69.0 ± 1.9 | 67.0 ± 2.0 | 68.7 ± 1.1 | 68.7 ± 1.1 |

*p<0.05 ANOVA followed by Tukey's HSD test compared to vehicle, same time point (n=6/group)

### EXAMPLE 5

Mob-5 mRNA in situ Hybridization on DRG Sections of Naive Rats

Non-radioactive in situ hybridisation methods are performed as described below in order to determine the expression pattern of Mob-5 in naïve (i.e without any surgical intervention) rat DRGs.

The cloned rat Mob 5 cDNA, as described in Example 1 with the cDNA sequence shown in Table 7, in pCR4 TOPO vector, was used for preparation of RNA probes for in situ hybridization. DNA is prepared using the QiaFilter maxiprep kit according to the manufacturer's instructions (Quiagen, West Sussex, UK) and linearised with Spel or NotI (New England Biolabs, Beverly, Mass.) for the antisense (positive) and sense (control) probes, respectively. DIG-labelled riboprobes are prepared from the linearised plasmids using T7 polymerase (antisense) or T3 polymerase (sense) with the DIG RNA labeling kit (Roche Diagnostics, Indianapolis, Ind.) as instructed by the manufacturer. An aliquot (4 μl) is checked by agarose gel electrophoresis to ensure there is no degradation and the rest is purified using a Quick Spin column (Roche Diagnostics) followed by overnight ethanol precipitation. The dried RNA pellet is resuspended in 50 μl 10 mM DTT solution and stored at −80° C.

TABLE 7
Nucleotide Sequence of Rat Mob 5 cDNA (SEQ ID NO: 11)

| | |
|---|---|
| ATGCAGACAA GCTTGAGACA ACAGATTCTC CCCGGCCTGA GCCTAATCCT | 50 |
| TCTCGTTTTG AGCCAAGTAC CAGAGCTTCA GGGTCAAGAG TTCCGATTTG | 100 |
| GGCCTTGCCA AGTGACCGGG GTGGTTCTCC CAGAACTGTG GGAGGCCTTC | 150 |
| TGGACTGTGA AGAACACTGT GAAAACTCAG GACGAGCTCA CAAGTGTCCG | 200 |
| GCTGTTGAAA CCACAGGTTC TGCAGAATGT CTCGGATGCC GAGAGCTGTT | 250 |
| ACCTTGCCCA CAGCCTGCTG AAGTTCTACT TGAACACTGT TTTCAAAAAC | 300 |
| TATCACAGCG AAATAGTCAA ATTCAAGGTC TTGAAGTCAT TCTCCACTCT | 350 |
| GGCCAACAAC TTTTTAGTCA TCATGTCCAA ACTGCAGCCT AGTAAGGACA | 400 |
| ATGCCATGCT TCCCATTAGT GACAGTGCAC GCCGGCGTTT TTTGCTGTTC | 450 |
| CACAGAACAT TCAAACAGTT GGACATAGAA GTGGCTTTGG CGAAAGCCTT | 500 |
| TGGGGAAGTG GACATTCTCC TGGCCTGGAT GCAGAATTTC TACCAGCTC | 549 |

Rats (Wistar) are killed by cervical dislocation and lumbar DRGs removed according to conventional methods. The tissues are mounted in Cryo-m-Bed (Bright, Huntington, UK) embedding compound, frozen on dry ice and stored at −80° C. DRG sections are cut to a thickness of 10 82 m using a Bright cryostat and thaw-mounted onto Superfrost polysine coated slides (BDH, Dorset, UK) and air dried.

Precautions for working with RNA are taken for the following procedures (DEPC treated solution, RNAse Zap (Ambion) treated bench and pipettes etc).

The slides are then prepared for hybridisation by serial incubation in:
1. 4% Paraformaldehyde—40 mins
2. 3×PBS—5 mins
3. 1×PBS—5 mins
4. dH₂O—30 secs
5. 0.1 M triethanolamine whilst stirring 25 l μ/ml acetic anhydride is added drop by drop and stirring continued for 10 mins
6. dH₂O—2 mins
7. dH₂O—30 secs
8. 50% formamide/3×SSC—5 mins
9. Slides are then air-dried in a fume hood

1 ml of hybridisation solution (50% deionised formamide, 5×SSC, 10 mM β mercaptoethanol, 10% dextran sulphate, 2 × Denhardt's solution, 250 μg/ml yeast tRNA and 500 μg/ml salmon sperm DNA, made prior to procedure, aliquoted and stored at −80° C.) is prewarmed to 65° C. For each slide to be hybridised, 150 μl of 1:100 probe in hybridisation buffer is heated to 65° C. 100 μl of probe/hybridisation buffer mix is pipetted onto a parafilm “coverslip” cut to the correct size that is subsequently picked up using the slide so that the sections are in contact with the mix. The slides are then incubated overnight at 55° C. in a chamber humidified to maximum saturation.

The parafilm coverslips are removed by incubation in pre-warmed (55° C.) 2×SSC/10 mM β-mercatoethanol. Post-hybridisation washes are carried out as follows:
1. 2×SSC/50% formamide/lmM EDTA pH 8.0/10 mM β-mercatoethanol at 55° C. for 30 minutes
2. 2×SSC at 37° C. for 30 minutes
3. 2×SSC/1 mM EDTA/20 μg/ml RNAse A/1 μg/ml RNAse T1 at room temperature for 40 minutes
4. 2×SSC/50% formamide/1 mM EDTA pH 8.0/10 mM β-mercaptoethanol at 55° C. for 30 minutes
5. 2×SSC/50% formamide/1 mM EDTA pH 8.0/10 mM β-mercaptoethanol at 55° C. for 30 minutes
6. 0.2×SSC at room temperature for 5 minutes

The slides are washed in 1×MAB (5×MAB stock solution is 0.5M Maleic Acid, 0.74M NaCl pH 7.5 with NaOH). The sections are encircled with a PAP pen (Sigma, St. Louis, Mo.). Blocking is performed in 2% BBR (Boehringer Blocking Reagent, Roche Diagnostics) diluted in 1×MAB/0.1% Tween 20 for one hour at room temperature in a chamber humidified to maximum saturation. This solution is replaced with Anti-Digoxigenin AP, Fab fragments (Roche Diagnostics) at a 1:100 concentration in 2% BBR in 1×MAB/0.1% Tween 20 and incubated at room temperature ovemight.

The slides are prepared for the colour reaction by washing twice in 1×MAB solution for 5 minutes each and once in freshly prepared NTMT (0.1M NaCl, 0.1M Tris HCl pH9.5, 50 mM MgCl2, 1% Tween 20, 2mM Levamisole Hydrochloride). The slides are dried and the colour reaction reagents (4.5 μl/ml 75 mg/ml NBT and 3.5 μl/ml 50 mg/ml BCIP (Gibco Life Technologies, Carlsbad, Calif.) in NTMT) added to section. The colour reaction is allowed to develop over several hours in the dark. Sections are mounted in PBS/glycerol (1:3) containing 25 mg/ml diazobicyclo-2,2-octane (DABCO) as an anti-fading agent. Images of antibody stained DRG sections are captured using a Nikon Eclipse 800 fluorescent microscope attached to a Hamamatsu cooled CCD camera and image capturing system. The software used for image capture is Image Pro Plus (Media Cybernetics, UK).

Results indicate that at lower levels of magnification (×4) high levels of Mob-5 expression are visible within the DRG neurons. At higher magnification (×10) strongest expression is seen in small and medium sized neurons. Lower levels of transcript are detected in large diameter neurons. Low levels of staining in non-neuronal cells, that is, glial cells, is observed. Hybridisation with the control sense probe results in no signal being detected.

### EXAMPLE 6

Antisense Oligonucleotides to Mob-5 Receptors (IL20 Alpha Receptor, IL 20 Beta Receptor, IL22 Receptor)

Antisense oligonucleotides (ASOs) useful to inhibit the expression of the Mob-5 receptor may be made and evaluated according to the methods described in Example 4.

### EXAMPLE 7

Oligoribonucleotides (siRNA's)and Antisense Oligonucleotides (ASO's)

Modified synthetic oligoribonucleotides and modified antisense oligonucleotides described in this invention are prepared using standard phosphoramidite chemistry on ABI394 or Expedite/Moss Synthesizers (Applied Biosystems) for in vitro use and on OligoPilot II (Amersham Pharmacia Biotech) for in vivo purpose. Phosphoramidites are dissolved in acetonitrile at 0.05 M concentration (0.2M on Oligopilot II), coupling is made by activation of phosphoramidites by a 0.2 M solution of benzimidazolium triflate in acetonitrile. Coupling times are usually comprised between 3-6 minutes. A first capping is made using standard capping reagents. Sulfurization is made by using a 0.05 M solution of N-ethyl, N-phenyl-5-amino-1,2,4-dithiazol-3-thione for two minutes (described in EP-A-0992506). Oxidation is made by a 0.1 M iodine solution in THF/Pyridine/Water (1:1:1) for two minutes. A second capping is performed after oxidation or sulfurization. Oligonucleotide growing chains are detritylated for the next coupling by 2% dichloroacetic acid in dichloromethane or dichloroethane. After completion of the sequences the support-bound compounds are cleaved and deprotected as “Tritylon” by a Methylamine solution (41% aqueous methylamine/33% ethanolic methylamine 1:1 v/v) at 35° C. for 6 h for oligoribonucleotides and by a 32% aqueous Ammonia solution at 55° C. for 16h for antisense oligonucleotides. Resulting suspensions are lyophilised to dryness. For oligoribonucleotides, 2′-O-silyl groups are removed upon treatment with 1 M tetrabutylammonium fluoride 10 min at 50° C. and 6 h at 35° C. The obtained crude solutions are directly purified by RP-HPLC. The purified detritylated compounds are analysed by Electrospray Mass spectrometry and Capillary Gel Electrophoresis and quantified by UV according to their extinction coefficient at 260 nM. The oligoribonucleotides and antisense oligonucleotides directed against rat MOB-5 and their controls are shown in Table 8.

TABLE 8

Oligoribonucleotides and ASO's directed against
rat MOB-5.

| | | |
|---|---|---|
| | Sequence | SEQ |
| Comment | (5′- to 3′) | ID NO: |

| | | |
|---|---|---|
| Antisense | UUC AGC AGG CUG UGG GCA | 12 |
| strand | AdGdG | |
| Sense strand | UUG CCC ACA GCC UGC UGA | 13 |
| | AdTdT | |
| 4 mismatch- | UUC CGA AGG CGG UGU GCA | 14 |
| antisense | AdGdG | |
| strand | | |
| 4 mismatch | UUG CAC ACC GCC UUC GGA | 15 |
| sense strand | AdTdT | |
| ASO | tca gcdA dGdGdC dTdGdT dGgg caa | 16 |
| ASO | aca gcTs CsTsCs GsGsCs Astc cga | 17 |
| ASO | tca gcAs GsGsCs TsGsTs Gsgg caa | 18 |
| ASO | tcc gaAs GsGsCs GsGsTs Gstg caa | 19 |
| ASO | GsGsCs CsAsTs CsCsAs csasgs | 20 |
| | tscsts tscst | |

RNAs NAS-11535 and NAS-11536, NAS-11537 and NAS-11538, are annealed together to give the siRNA's. The ASO NAS-4660 targets an unrelated gene. Unless otherwise stated, internucleotidic linkages are phosphodiester, N=ribonucleoside, dN=deoxyribonucleoside, n=2′-O(methoxyethyl) ribo-nucleoside, s=phosphorothioate.

### EXAMPLE 8

Effects of MOB-5 siRNA on Mechanical Hyperalgesia in Rats with Neuropathic Pain (Seltzer Model)

Four groups of rats were ligated on the left hind limb on day 0 and base line mechanical hyperalgesia was measured. An additional unligated group (naïve) was set up as control. Rats were cannulated on day 10 and infused with vehicle, RNAi or missense for a further 6 days. Paw withdrawal thresholds (left paw) were measured daily (Table 9). Vehicle: isotonic buffer, n=8/treatment group. The right paw for each group were also measured but showed no difference in paw withdrawal threshold to naïve animals.

TABLE 9

| | Paw withdrawal thresholds (g) | | | |
|---|---|---|---|---|
| | | Neuropathic | | |
| | | | MOB-5 RNAi | |
| | Naive | | missense | MOB-5 RNAi |
| Day | vehicle | vehicle | 400 μg | 400 μg |
| 0 | 101.4 ± 2.4 | 103.1 ± 1.3 | 99.4 ± 1.1 | 100 ± 1.9 |
| — | | | | |
| 10 | 101.4 ± 1.8 | 60.6 ± 1.5 | 62.5 ± 1.3 | 60 ± 1.6 |
| 11 | 102.1 ± 1.5 | 61.9 ± 1.3 | 66.2 ± 1.6 | 67.5 ± 2.1 |
| 12 | 102.1 ± 2.1 | 62.5 ± 0.9 | 65.5 ± 1.1 | 75 ± 2.1 |
| 13 | 105 ± 2.2 | 63.7 ± 1.5 | 66.9 ± 1.6 | 81.2 ± 2.9 |
| 14 | 102.1 ± 2.1 | 64.4 ± 1.5 | 62.5 ± 2.3 | 78.7 ± 4.2 |
| 15 | 102.1 ± 1.8 | 62.5 ± 0.94 | 61.9 ± 2.3 | 74.4 ± 2.7 |
| 16 | 102.1 ± 1.5 | 62.5 ± 0.94 | 58.7 ± 2.3 | 75 ± 2.3 |

### EXAMPLE 9

Effects of MOB-5 siRNA on Mechanical Allodynia in Rats with Neuropathic Pain (Seltzer Model)

Four groups of rats were ligated on the left hind limb on day 0 and base line mechanical allodynia was measured. An additional unligated group (naïve) was set up as control. Rats were cannulated on day 10 and infused with vehicle, RNAi or missense for a further 6 days. Von Frey thresholds on the left paw were measured daily (Table 10). Vehicle: isotonic buffer, n=8/treatment group. The right paw for each group were also measured but showed no difference in paw withdrawal threshold to naïve animals.

TABLE 10

| | Von Frey thresholds (g) | | | |
|---|---|---|---|---|
| | | Neurophathic | | |
| | | | MOB-5 RNAi | |
| | Naive | | missense | MOB-5 RNAi |
| Day | vehicle | vehicle | 400 μg | 400 μg |
| 0 | 14.3 ± 0.7 | 12 ± 1.2 | 11.9 ± 0.9 | 12.9 ± 1.0 |
| — | | | | |
| 10 | 15 ± 0 | 5.9 ± 1.5 | 3 ± 0.6 | 6.1 ± 1.5 |
| 11 | 14.3 ± 0.7 | 5.5 ± 1.1 | 2.7 ± 0.5 | 5.7 ± 0.9 |
| 12 | 14.3 ± 0.7 | 4.2 ± 1.2 | 2.5 ± 0.5 | 6.2 ± 0.8 |
| 13 | 14.3 ± 0.7 | 4.2 ± 1.2 | 2.7 ± 0.5 | 5.7 ± 0.8 |
| 14 | 15 ± 0 | 4.0 ± 1.0 | 2.5 ± 0.3 | 6.7 ± 0.8 |
| 15 | 12.1 ± 1.0 | 3.7 ± 0.7 | 2.5 ± 0.3 | 6.5 ± 0.7 |
| 16 | 12.1 ± 1.0 | 4.2 ± 0.7 | 3.2 ± 0.6 | 6.7 ± 0.7 |

1. A method to treat or ameliorate chronic pain comprising administering to a subject in need thereof an effective amount of a Mob-5 modulator or a pharmaceutical composition comprising an effective amount of a Mob-5 modulator.

2. The method of claim 1 wherein said chronic pain is chronic neuropathic pain.

3. The method of claim 1 wherein said Mob-5 modulator inhibits or stimulates the activity of Mob-5 in said subject.

4. The method of claim 1 wherein said Mob-5 modulator inhibits or stimulates Mob-5 gene expression in said subject.

5. The method of claim 1 wherein said modulator comprises any one or more substances selected from the group consisting of antisense oligonucleotides, triple helix DNA, ribozymes, RNA aptamers, siRNA and double stranded RNA wherein said substances are designed to inhibit Mob-5 gene expression.

6. The method of claim 1 wherein said modulator comprises one or more antibodies to Mob-5, or fragments thereof, wherein said antibodies or fragments thereof can inhibit Mob-5 activity.

7. The method of claim 1 wherein said modulator comprises one or more agonists to the Mob-5 receptor.

8. The method of claim 1 wherein said modulator comprises one or more antagonists to Mob-5.

9. A method to identify modulators useful to treat or ameliorate chronic pain comprising assaying for the ability of a candidate modulator to inhibit or stimulate Mob-5 activity.

10. A method to identify modulators useful to treat or ameliorate chronic pain comprising assaying for the ability of a candidate modulator to inhibit or stimulate Mob-5 gene expression.

11. The method according to claim 9 or 10 wherein said method further comprises assaying for the ability of an identified modulator to reverse the pathological effects observed in animal models of chronic pain and/or in clinical studies with subjects with chronic pain.

12. A pharmaceutical composition comprising a Mob-5 modulator in an amount effective to treat or ameliorate chronic pain in a subject in need thereof, optionally mixed with a pharmaceutically acceptable carrier.

13. The pharmaceutical composition of claim 12 wherein said modulator comprises one or more antibodies to Mob-5, or fragments thereof, wherein said antibodies or fragments thereof inhibit Mob-5 activity.

14. The pharmaceutical composition of claim 12 wherein said modulator comprises one or more agonists or antagonists to the Mob-5 receptor.

15. A method to diagnose subjects suffering from chronic pain who may be suitable candidates for treatment with Mob-5 modulators comprising assaying mRNA levels of this protein in a biological sample from said subject wherein subjects with increased levels compared to controls would be suitable candidates for Mob-5 modulator treatment.

16. A method to diagnose subjects suffering from chronic pain who may be suitable candidates for treatment with Mob-5 modulators comprising detecting levels of this protein in a biological sample from said subject wherein subjects with increased levels compared to controls would be suitable candidates for Mob-5 modulator treatment.

17. A method to treat or ameliorate chronic pain comprising:
(a) assaying for Mob-5 mRNA and/or protein levels in a subject; and,
(b) administering to a subject with increased levels of Mob-5 mRNA and/or protein levels compared to controls a Mob-5 modulator in an amount sufficient to treat or ameliorate the pathological effects of chronic pain.

18. A diagnostic kit for detecting mRNA levels and/or protein levels of Mob-5 in a biological sample, said kit comprising:
(a) a polynucleotide of Mob-5 or a fragment thereof;
(b) a nucleotide sequence complementary to that of (a);
(c) a Mob-5 polypeptide, or a fragment thereof; or (d) an antibody to a Mob-5 polypeptide
wherein components (a), (b), (c) or (d) may comprise a substantial component.

19. A kit for administering a pharmaceutical composition comprising a Mob-5 modulator in an amount effective to treat or ameliorate chronic pain in a subject in need thereof, wherein said modulator comprises one or more antibodies to Mob-5, or fragments thereof, and wherein said antibodies or fragments thereof inhibit Mob-5 activity, optionally mixed with a pharmaceutically acceptable carrier.

20. A gene therapy vector comprising a nucleic acid molecule that encodes Mob-5 or a biologically active fragment thereof.

21. A nucleic acid molecule that is complementary to a nucleic acid molecule that encodes Mob-5 or a fragment thereof.

2. (canceled)

23. A method to treat or ameliorate chronic pain comprising administering to a subiect in need thereof an effective amount of a monoclonal antibody which specifically binds an epitope of Mob-5 or a biologically active fragment thereof.

24. (canceled)

25. A method to treat or ameliorate chronic pain comprising administering to a subiect in need thereof an effective amount of a compound selected from the group comprising antisense molecules, ribozyme, and a nucleic acid molecules which promote triple helix formation, that specifically inhibit the expression of Mob-5 genes.

26. The method according to claim 10 wherein said method further comprises assaying for the ability of an identified modulator to reverse the pathological effects observed in animal models of chronic pain and/or in clinical studies with subjects with chronic pain.
